(19) United States
(12) Patent Application Publication
(10) Pub. No.: US 2011/0137394 A1
(43) Pub. Date: Jun. 9, 2011

(54) METHODS AND SYSTEMS FOR PENETRATING ADJACENT TISSUE LAYERS

(75) Inventors: John Lunsford, San Carlos, CA (US); Kenneth F. Binmoeller, Rancho Santa Fe, CA (US)

(73) Assignee: XLumena, Inc., Mountain View, CA (US)

(21) Appl. No.: 12/790,553

(22) Filed: May 28, 2010

Related U.S. Application Data
(60) Provisional application No. 61/182,319, filed on May 29, 2009.

(51) Int. Cl. A61F 2/82; A61M 5/32; A61B 18/18

(52) U.S. Cl. 623/1.11; 604/272; 606/45

(57) ABSTRACT

Penetration and dilation of passages from a first body lumen to a second body lumen are achieved while providing tension anchoring of the luminal walls to inhibit the leakage of body fluids. In one embodiment, one or more T-bar anchors may be used to provide the tensioning of the body lumen walls. In a second embodiment, a plurality of hooked or everted wires may be provided on a catheter which is used to penetrate and dilate a passage between the luminal walls.

### CROSS-REFERENCES TO RELATED APPLICATIONS

This application claims the benefit of provisional application 61/182,319 (Attorney Docket No. 026923-001500US), filed on May 29, 2009, the full disclosure of which is incorporated herein by reference. This application is also related but does not claim the benefit of commonly owned copending application nos. 12/427,215 (Attorney Docket No. 026923-000710US), filed on Apr. 21, 2009; 12/757,408 (Attorney Docket No. 026923-001210US), filed on Apr. 9, 2010, 12/757,421 (Attorney Docket No. 026923-001310US), filed on Apr. 9, 2010; and 12/772,762 (Attorney Docket No. 026923-001410US), filed on May 3, 2010, the full disclosures of which are incorporated herein by reference.

### BACKGROUND OF THE INVENTION

1. Field of the Invention

The present invention relates generally to medical methods and apparatus. More particularly, the present invention relates to methods and apparatus for penetrating adjacent tissue layers, for example with a dilator, which can be used to deliver a stent or other tissue approximating device.

A number of inter and intra-luminal endoscopic procedures require precise placement of anchors or stents. For example, a number of procedures may be performed by entering the gastrointestinal (GI) tract through a first organ or structure, such as the esophagus, stomach, duodenum, small intestine, or large intestine, and delivering the anchor or stent to adjacent organs and lumen or tissue structures such as an adjacent portion of the GI tract, the bile duct, the pancreatic duct, the gallbladder, the pancreas, cysts, pseudocysts, abscesses, and the like. While primarily intended for use in the GI tract, the methods and apparatus can be used for access to and from portions of the urinary tract, such as the urinary bladder and ureter, the pulmonary tract, such as the trachea and bronchi, and biliary such as the biliary tract, such as the bile duct and gallbladder, as well.

Intra-ductal stents are commonly used to facilitate the opening of closed vessels for access, drainage or other purposes. Tissue anchors are used to secure adjacent tissues or organs. Inter-luminal tissue anchors, which include a central lumen, are used to facilitate fluid communication between adjacent ducts, organs or lumens. Often, the precise placement of the tissue anchor or stent is necessary, especially when the tissue anchor or stent has well defined anchoring elements at the proximal and/or distal ends, the device is used to secure adjacent lumens.

When deploying a stent or other tissue anchor between adjacent body lumens, organs, or other structures, it is typically necessary to penetrate both a wall of the first body lumen through which access is established and a wall of a second body lumen which is the target for the procedure. When initially forming such access penetrations, there is a significant risk of leakage from either or both of the access body lumen and the target body lumen. In some procedures, such as those involving transgastric or transduodenal bile duct access, loss of body fluid into surrounding tissues and body cavities can present a substantial risk to the patient. The risk can be exacerbated when it is necessary to not only penetrate the luminal walls to gain initial access, usually with a needle, but to subsequently enlarge or dilate the initial penetration, for example by passing a tapered dilator and/or dilating balloon over the needle used to establish initial access.

Thus, it would be desirable to establish initial luminal wall penetrations and optional dilation in order to deploy a stent, anchor, or for other purposes, while minimizing the risk of body fluid leakage. It would be further desirable to provide improved protocols and access tools which are capable of being deployed from endoscopes present in a first body lumen to access adjacent body lumens or cavities while minimizing the risk of leakage. Such access tools and protocols should be compatible with a wide variety of procedures, such as placement of stents or other tissue anchors between adjacent luminal walls, and will preferably reduce or eliminate the need to exchange tools during the access procedure. It would be further desirable if tools and access protocols could be provided which allow for the continuous application of tension on the luminal walls to maintain said walls in close apposition during the stent or anchor placement or other procedure in order to reduce the risk of body fluid loss during most or all stages of the procedure. At least some of these objectives will be met by the inventions described below.

2. Description of the Background Art

US2009/0281379 and US2009/0281557 describe stents and other tissue anchors of the type that can be deployed by the apparatus and methods of the present invention. The full disclosures of these publications are incorporated herein by reference. US 2003/069533 describes an endoscopic transduodenal biliary drainage system which is introduced through a penetration, made by a trans-orally advanced catheter having a needle which is advanced from the duodenum into the gallbladder. U.S. Pat. No. 6,620,122 describes a system for placing a self-expanding stent from the stomach into a pseudocyst using a needle and an endoscope. US 2005/0228413, commonly assigned with the present application, describes a tissue-penetrating device for endoscopy or endosonography-guided (ultrasonic) procedures where an anchor may be placed to form an anastomosis between body lumens, including the intestine, stomach, and gallbladder. See also U.S. Pat. No. 5,458,131; U.S. Pat. No. 5,495,851; U.S. Pat. No. 5,944,738; U.S. Pat. No. 6,007,522; U.S. Pat. No. 6,231,587; U.S. Pat. No. 6,655,386; U.S. Pat. No. 7,273,451; U.S. Pat. No. 7,309,341; U.S. Pat. No. 2004/0243122; US 2004/0249985; US 2007/0123917; WO 2006/062996; EP 1314404 Kahaleh et al. (2006) Gastrointestinal Endoscopy 64:52-59; and Kwan et al. (2007) Gastrointestinal Endoscopy 66:582-586. Shaped balloons having differently sized segments and segments with staged opening pressures are described in U.S. Pat. Nos. 6,835,189; 6,488,653; 6,290,485; 6,022,359; 5,843,116; 5,620,457; 4,990,139; and 3,970,090.

### BRIEF SUMMARY OF THE INVENTION

The present invention provides methods and apparatus for establishing transluminal access by penetrating and optionally dilating passages between a first body lumen and a second body lumen. Such transluminal access may be intended for any medical purpose but will usually be intended for performing transluminal therapeutic endoscopy where the first body lumen is typically within the gastrointestinal (GI) tract, including the esophagus, the stomach, the duodenum, the small intestines, and the large intestines. The second body lumen, which is the target of the access, will typically be an organ or other tissue structure which lies adjacent to the gastrointestinal tract (or may be another part of the GI tract), including the bile duct, the pancreatic duct, the gallbladder, cysts, pseudocysts, abscesses, the pancreas, the liver, the urinary bladder, the duodenum, jejunum, and colon. Particular procedures which may benefit from the access methods and apparatus of the present invention include gastrojejunostomy, gastroduodenostomy, and gastrocolostomy. Other procedures which can benefit from the methods and apparatus of the present invention include vascular bypass including porto systemic shunts and transjugular intrahepatic portasystemic shunt (TIPS) procedures.

The methods and apparatus of the present invention are advantageous in a number of ways. In particular, the methods and apparatus provide for a substantially continuous apposition of the luminal walls to be penetrated and/or dilated to reduce the risk of body fluid leakage into body cavities surrounding the lumens. The wall apposition is achieved without interfering with the primary penetration and/or dilation by locating one or more tension anchors across the luminal walls at locations which are laterally displaced or spaced-apart from a target location through which the penetration has been or will be formed. The tension anchors may be deployed from the endoscope separately from the tool(s) used to form and optionally dilate the primary luminal wall penetration. In other embodiments, the tension anchor(s) will be deployed from the same tool which is used to form, dilate, and optionally place a stent or other tissue anchor in the penetration. In all cases, deploying the tension anchor on a posterior surface of the luminal wall of the second (target) body lumen provides and maintains tension to hold the luminal walls in apposition while the walls are penetrated and/or the penetration is dilated and optionally a stent/anchor is deployed in the penetration.

The stents and anchors which may optionally be deployed by the methods and apparatus of the present invention will typically have distal and proximal flange elements which, at the end of the implantation procedure, will engage the luminal walls and hold the luminal walls together. In addition, the flanges and stent/anchor will seal sufficiently against the luminal walls to inhibit leakage from the time of their initial deployment. Usually, the stent/anchors will include or define a central opening or passage to allow the exchange of fluid between the first body lumen and the second body lumen, often being suitable for drainage of fluid from the second body lumen into the first body lumen, e.g., for gallbladder or bile duct drainage. A number of suitable stent/anchors and tools for their deployment are described in co-pending applications US 2009/0281557 and US 2009/0281379, the full disclosures of which are incorporated herein by reference.

In a first aspect of the present invention, a method for penetrating and optimally advancing a dilator distally through apposed luminal walls of adjacent first and second body lumens at a target site on an anterior surface of a first luminal wall comprises deploying one or more tension anchor(s) through the apposed luminal walls and drawing the tension anchor proximally to hold a posterior surface of the first luminal wall against an anterior surface of a second luminal wall. The methods may employ a single tension anchor but will more typically employ a plurality of such tension anchors. In all cases, the tension anchor(s) will be deployed at location(s) which are laterally offset from the target site which is to be penetrated and optionally dilated. By drawing proximally on the tension anchor(s) and holding the luminal walls together, leakage of body fluid from either or both of the first and second body lumens will be substantially inhibited. In some cases, the tension anchor(s) will be deployed prior to any other penetrations through the luminal walls. In other instances, an initial needle penetration may be formed through the luminal walls with the tension anchor(s) being deployed after the initial penetration and prior to advancement of a dilator or other tools or instruments over the needle.

In a first specific embodiment of the methods of the present invention, deploying the tension anchors comprises advancing a tether having a self-deploying anchor from the first body lumen, through the apposed luminal walls at said laterally offset locations, and into the second body lumen. The anchors, which may be conventional T-bar anchors (T-tags) delivered by a needle located through an endoscope, will deploy within the second body lumen so that they engage the posterior surface of the second luminal wall so that by drawing on the tether, tension can be placed on the second luminal wall to draw the first and second luminal walls together. The deployment of such self-deploying anchors can be performed prior to any other tissue penetrations or subsequent to an initial needle penetration, as generally described earlier.

In a second specific embodiment of the methods of the present invention, deploying the tension anchor will comprise positioning a distal end of a catheter within the first body lumen at the target site and advancing one or more tension anchor(s) from the catheter through the apposed luminal walls. Typically, the catheter will be placed through an endoscope which can provide both viewing and steering capabilities. The catheter will carry at least one tension anchor and will optionally carry a plurality of tension anchors, typically being adapted to axially reciprocate through lumens or passages arranged peripherally or circumferentially about the exterior of the catheter.

In the exemplary embodiments, the catheter-deployed tension anchors will comprise a wire having a pre-shaped distal end where the wire can be advanced from an axial passage on or in the catheter, and the distal end assumes a three-dimensional geometry or shape within the second body lumen. In some instances, the three-dimensional shape may be a simple hook or other everting structure, and the change in shape can be the result of release from constraint or of inducing a shape memory change, for example, by heating or passing a current through a wire composed of a shape-memory alloy to transition the alloy from its initial straight configuration to the three-dimensional configuration. In all instances, once the shape transition has occurred, the wire may be drawn proximally against the posterior surface of the second luminal wall to apply tension and draw the second luminal wall against the first luminal wall to inhibit body fluid leakage. Use of catheter for deploying single or multiple tension anchors is advantageous since the same catheter can be used for performing tissue penetrations, dilations, anchor placement, stent placement, and other protocols without the need to exchange tools.

Deployment of the tension anchors from the catheter may be performed either before or after initial penetration with an access needle. In all instances, however, the tension anchor(s) will be deployed from the catheter prior to advancement of a dilator from the catheter, typically over a previously deployed access needle. Advancement of the dilator can also be used to release a self-expanding stent or other tension anchor within the passage or penetration which has just been enlarged by the dilator. Preferred stents are described in the copending applications incorporated above and have flanges which will hold the first and second luminal walls together even after the dilator and tension anchors are removed.

In a second aspect of the present invention, an apparatus for dilating a passage through apposed luminal walls comprises a catheter, a needle, a dilator, and at least one tension anchor. The catheter has a proximal end, a distal end, and a central passage therethrough. The needle is reciprocatably disposed within the passage in the catheter and has a tissue penetrating tip. In this way, the tissue penetrating tip can be advanced through tissue as the needle is deployed distally from the central passage of the catheter. The dilator is slidably mounted over the needle, with the needle typically being coaxially received within a central passage in the dilator. In this way, the dilator can be advanced over the needle to dilate the penetration formed by the needle through the apposed luminal walls. The dilator will have a tapered distal or front end and will optionally include one or more blades for cutting the tissue as the dilator is advanced through the penetration. The tension anchor(s) are reciprocatably mounted on the catheter so that they can be advanced to penetrate tissue location(s) which are laterally offset from the dilator/needle penetration location. As discussed above, having such laterally offset tension anchor(s) allows the luminal walls to be tensioned and brought together while leaving the access area free for penetration, dilation, and placement of stents, anchors, and other implantable devices.

In specific examples of the apparatus of the present invention, the tension anchor(s) will be disposed in one or more peripheral lumens disposed on or over an exterior surface of the catheter. Usually, only one tension anchor will be disposed in each peripheral lumen, but optionally two or more could be included in individual lumens. Exemplary tension anchor(s) for use with the catheter embodiments of the present invention comprise a wire having a pre-shaped distal end. The distal end will be straightened when present in the peripheral lumen and advanced through the apposed luminal walls and will be adapted to assume a three-dimensional geometry when present in the second body lumen beyond the second luminal wall. Typically, the wire will be adapted to assume a hook or other everted structure which can be drawn proximally to engage the tissue layers and apply tension thereto. The hook will be made of a shape memory alloy pre-formed to the hooked or curved geometry and constrained in a straightened shape prior to being deployed. Deploying the tension anchor beyond a pre-determined distance will remove constraint from the non-straight memory formed section of the tension anchor and cause the tension anchor the take a pre-shaped three-dimensional configuration.

In a specific embodiment intended to facilitate stent delivery, the dilator comprises a shaft having a tapered dilating tip at its distal end. The dilating tip may optionally have a sharpened blade and/or an electrosurgical tip to cut tissue as it is advanced through the luminal walls. The electrosurgical tip can provide for both cutting and coagulation when the proper radiofrequency wave form is applied. A self-expanding stent can be carried on the dilator shaft proximal of the tapered tip where the stent is constrained on the shaft, optionally by a retractable sheath but usually by the catheter itself. Thus, after the tissue penetration in the apposed luminal walls has been formed, the constraint can be removed to deploy the stent in the dilated penetration.

### BRIEF DESCRIPTION OF THE DRAWINGS

FIG. 1 is a perspective view of a tension anchor delivery tool useful in the methods of the present invention.

FIG. 2 is a detailed view of the distal end of the tool of FIG. 1 showing a self-deploying distal end of a tension anchor in place in the tool.

FIGS. 3A-3H illustrate use of the tool of FIGS. 1 and 2 for placing the tension anchor between apposed luminal walls and using said anchor for making a penetration, dilating the penetration, and placing a stent in said dilated penetration, all while tension is maintained on the tension anchor.

FIG. 4 illustrates a catheter having integral tension anchors and deployable tools for penetrating and dilating luminal walls of adjacent body lumens.

FIGS. 5A-5I illustrate use of the tool of FIG. 4 for penetrating, applying tension, and delivering a stent to apposed luminal tissue walls in accordance with the principles of the present invention.

### DETAILED DESCRIPTION OF THE INVENTION

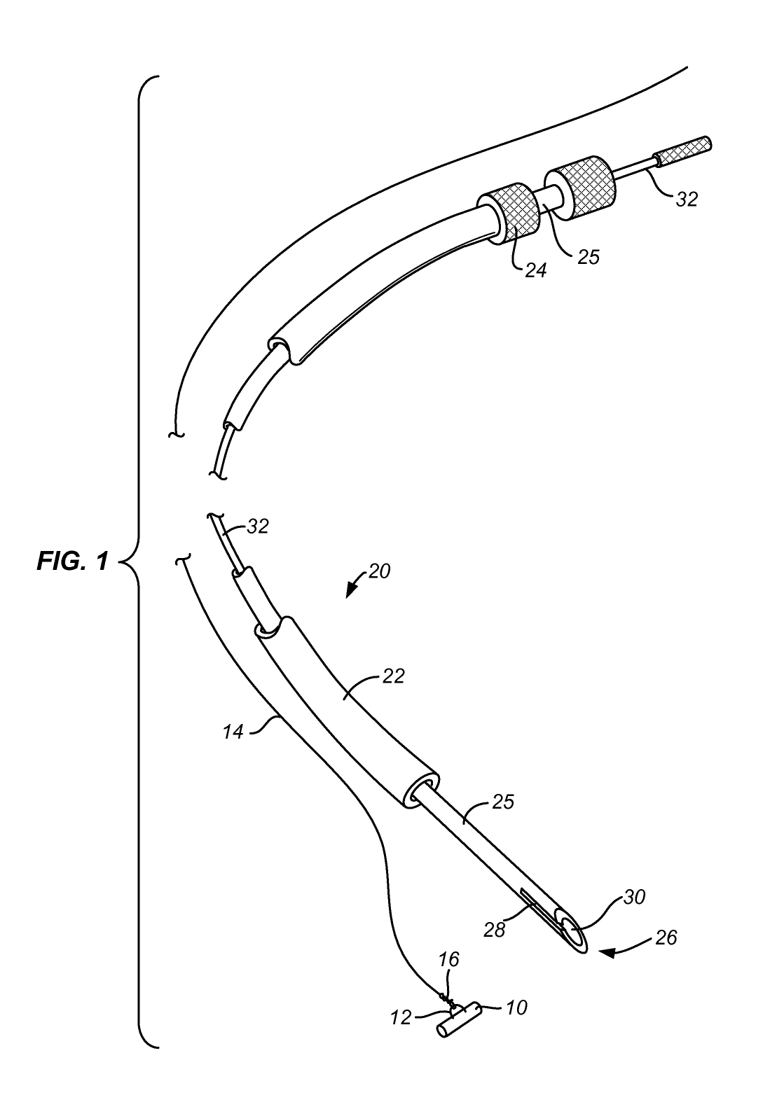

T-tags, also called T-bar anchors, useful in the present invention comprises anchoring devices which include a T-anchor 10 with an attachment loop 12, a tethering suture 14 connected to the T-anchor through the attachment loop with a knot 16 as in FIG. 1. T-tags are placed using a penetrating assembly 20, typically a standard 19 gauge endoscopic needle assembly, including a needle sheath 22, a handle 24, a needle 25 with sharpened distal tip 26 and a slit 28 and opening 30 in the distal end of the needle to accommodate the T-anchor 10 and tethering suture 14. A stylette 32 is a wire like structure positioned lengthwise through the central lumen of the 19 gauge endoscopic needle that is used to push and deploy the T-tag into the target lumen. The aforementioned assembly allows the needle 25, loaded with a T-anchor 10, to be thrust through one or several layers of tissue followed by deployment of the T-anchor in a distal tissue surface, removal of the needle and securing the tether suture proximally, thereby holding the tissues in apposition as a tension is maintained on the needles in a posterior direction.

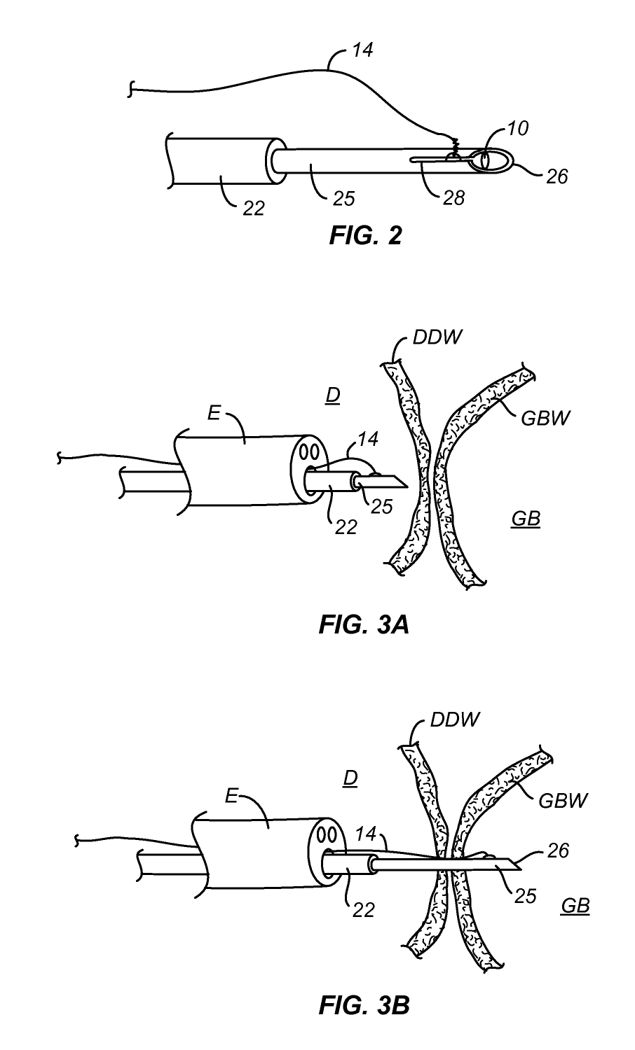

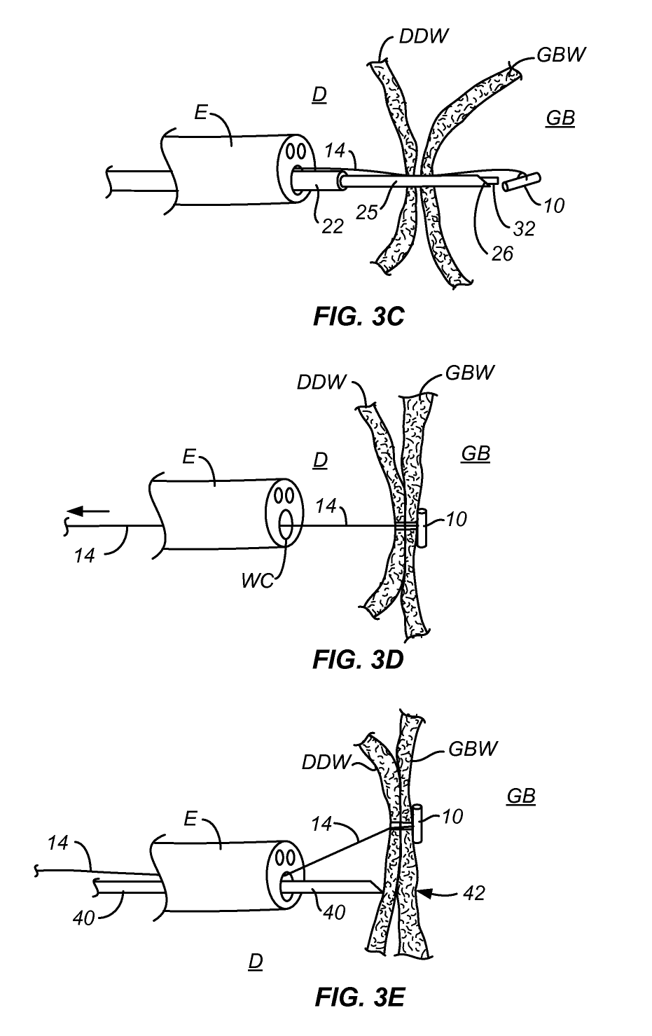

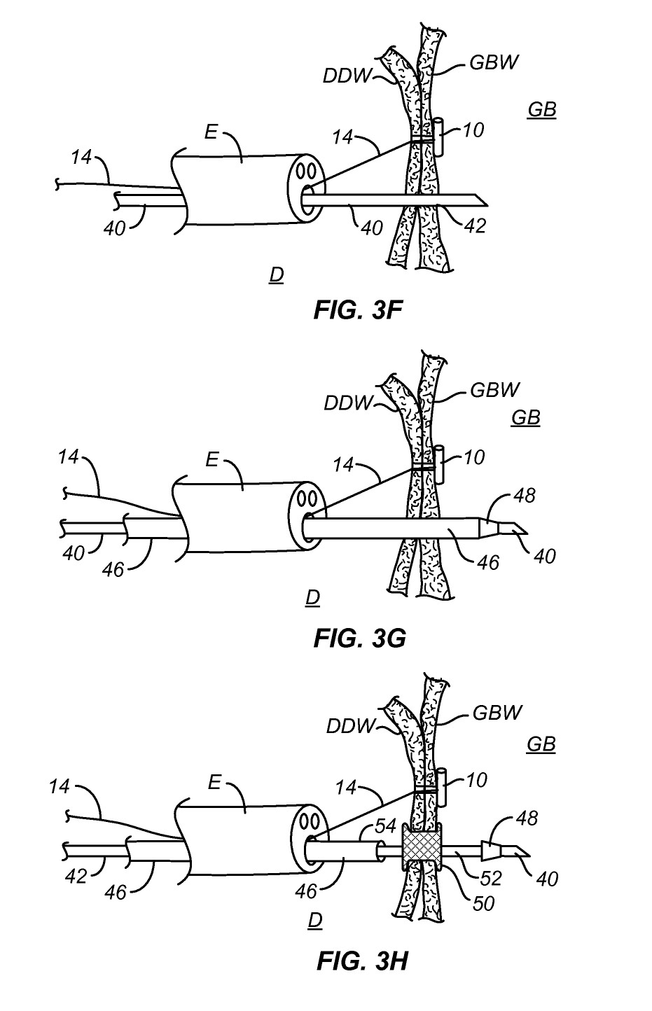

Following endoscopic identification of the target gallbladder, a T-tag 10 loaded in the 19 gauge needle 25 (FIG. 2), is advanced through the working channel of an endoscope E, and the needle tip 26 placed in a target location adjacent to the duodenal wall DDW in the duodenum D and transluminally adjacent to the gallbladder GB, as shown in FIG. 3A. The needle 25 is then advanced through the luminal wall tissue placing the distal section of the needle inside the gallbladder as in FIG. 3B. The T-anchor 10 is then deployed by advancing the stylette 32 in the distal direction and pushing the T-anchor out of the tip 26 of needle 25 as in FIG. 3C.

Once the T-anchor 10 is deployed in the gallbladder GB lumen, the needle 25 is removed from the endoscope E with the tethering suture 14 extending through the endoscope working channel WC, exiting at the proximal end. The tethering suture 14 can then be secured, with mild tension to maintain the gallbladder wall GBW in close apposition to the duodenum wall DDW, thereby preventing bile leakage into the peritoneal cavity as shown in FIG. 3D.

One, two or several T-tags can be placed and, if desired, the endoscope E can be removed allowing the sutures to exit the mouth of the patient, while always holding mild tension on the tethering sutures. Alternately a locking pledget (not shown) can be advanced over the suture, and locked with mild tension at the wall of the duodenum, with excess suture being removed, this holding the structures together.

Following apposition of the gallbladder wall GBW to the duodenum wall DDW with T-tags 10 as shown in FIG. 3D, a new 19 gauge needle 40 is used to make a penetration from the duodenum D into the gallbladder GB at a location 42 laterally spaced from the T-tags 10 as in FIG. 3E. The needle 42 may then be advanced across the duodenum wall DDW and the gallbladder wall GBW while the tether maintains tension on the walls via the T-tag anchor 10, as shown in FIG. 3F. The tension allows the needle 42 to pass with reduced risk of body fluid leakage.

A principal reason for providing tension using tether 14 and T-tag 10, however, is the desire to pass a larger diameter dilator 46 across the luminal walls DDW and GBW, as shown in FIG. 3G. Whereas the 19-gauge needle will typically have a diameter of about 1 mm, the dilator 46 will usually have a diameter in the range from 2 mm to 5 mm. Even though the dilator has a tapered entry tip 48 to facilitate passage through and enlargement of the penetration caused by needle 40, such a large diameter will still have a strong tendency to separate the luminal walls DDW and GBW, increasing the risk of body fluid loss. The presence of the tension anchors comprising the T-tag 10 and suture tether 14, however greatly reduced the risk that the luminal walls will separate and allow for leakage. Moreover, in many cases, a plurality of tension anchors, often two, many times three, and sometimes four or more, can be used to better bring the luminal walls into apposition and reduce the risk of body fluid leakage.

Although the dilator and resulting enlarged penetration can be used for a variety of purposes and protocols, they will most often be used to deliver a stent 50, as illustrated in FIG. 3H, for example, the stent 50 may be self-expanding and carried over a shaft 52 of the dilator 46. By initially constraining the self-expanding stent 50 within a retractable sheath 54 of the dilator 46, the sheath can be retracted to release the stent 50 and allow it to expand within the dilated luminal wall penetration provided by the dilator.

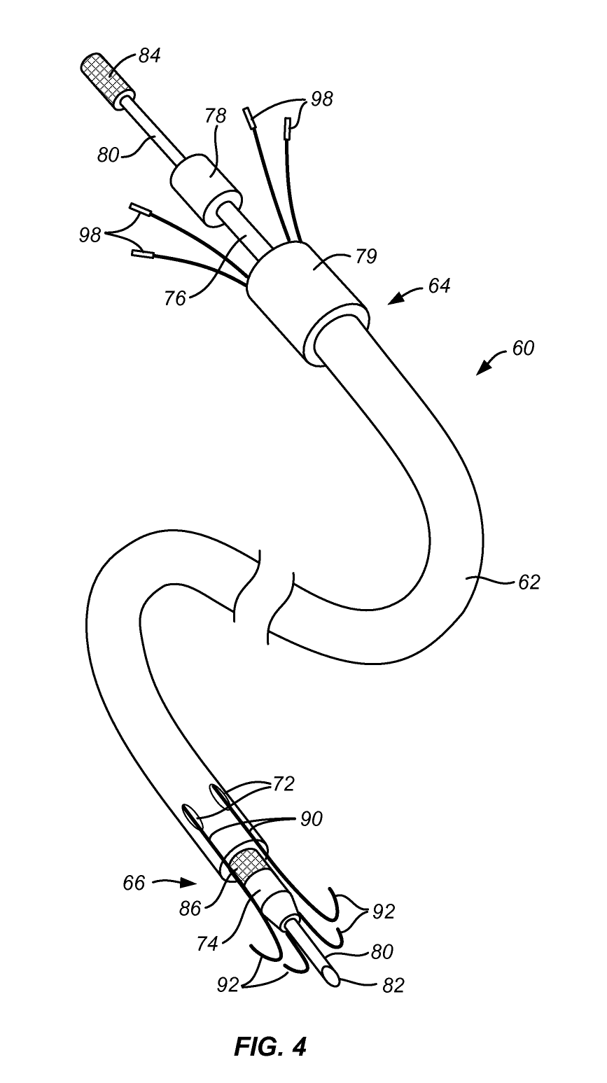

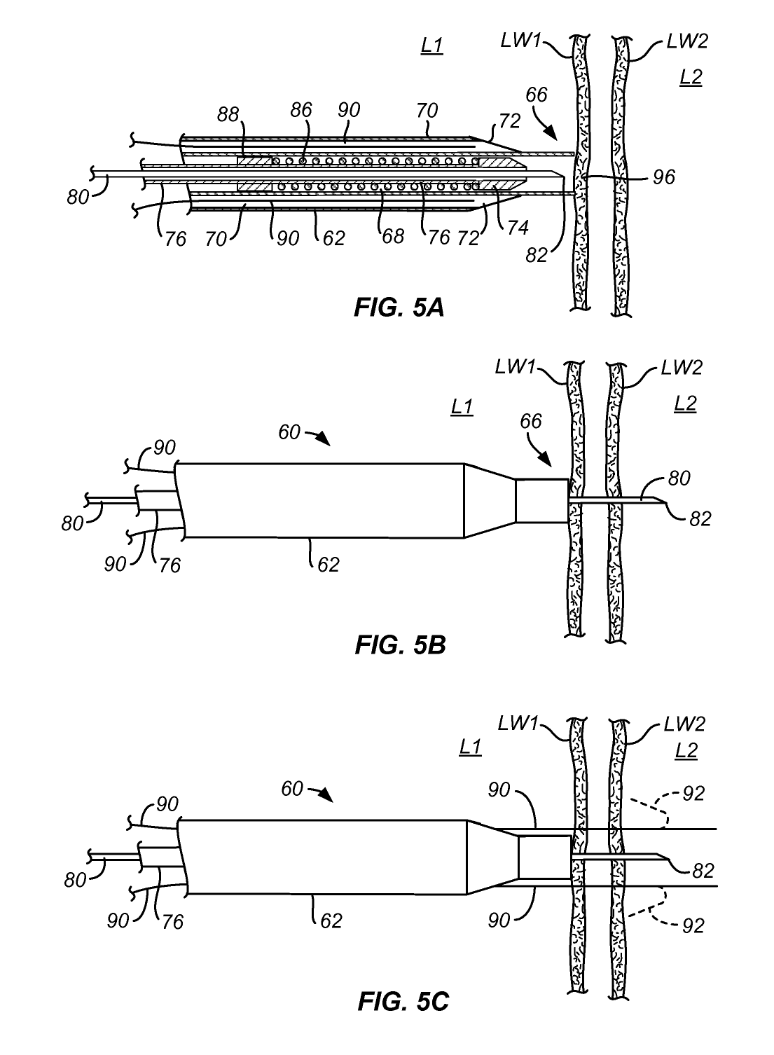

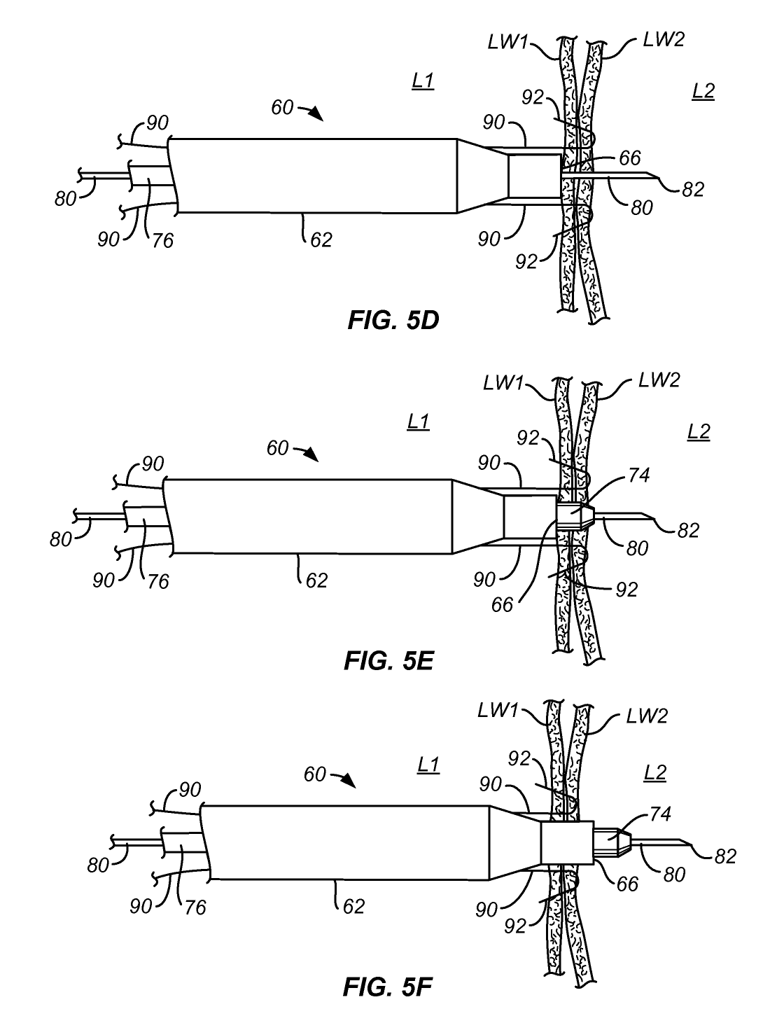

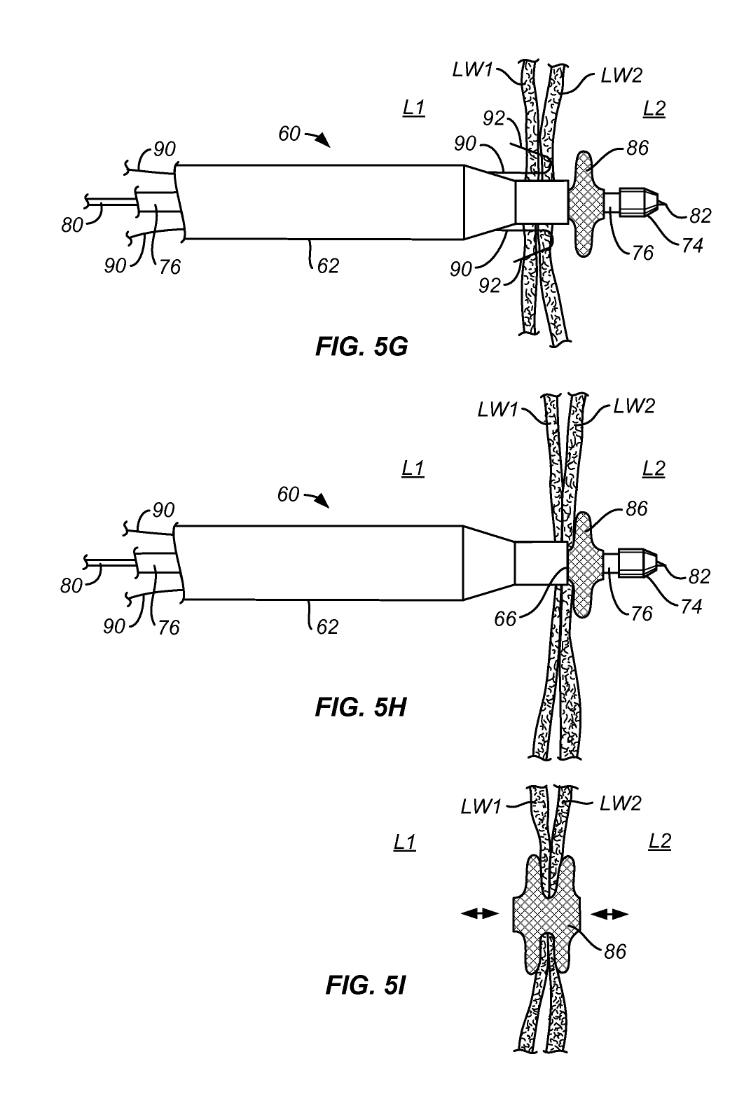

Referring now to FIG. 4, an alternative apparatus for performing the methods of the present invention is illustrated. A catheter 60 comprises a tubular body 62 having a proximal end 64 and a distal end 66. As best seen in FIG. 5A, the tubular body 62 has a center passage 68 and a plurality (four as illustrated) of peripheral passages 70 disposed in parallel to the center passage 68. Each of the peripheral passages 70 terminates in a distal peripheral port 72.

A dilator assembly comprising a tapered dilator tip 74 disposed at the distal end of a hollow shaft 76 is received in the center passage 68 of the tubular body 62, as seen in FIGS. 4 and 5A. The dilator assembly will be reciprocatably mounted so that the tapered dilator tip 74 may extend beyond the distal end 66 of the tubular body 62 and central passage 68, as seen in FIG. 4. The dilator assembly further includes a proximal grip 78 at its proximal end to permit manipulation by the user. In particular, the user can advance the dilator assembly by holding the grip 78 in one hand and a corresponding proximal grip 79 at the proximal end of the tubular body 62 of catheter 60 in the other hand. In this way, a user can easily manually advance and retract the dilator assembly within the center passage 68 of the catheter by moving the grips apart or together, respectively.

A needle 80, typically a 19 gauge endoscopic needle, is reciprocatably disposed within the hollow center lumen of the hollow shaft 76 of the dilator assembly, as best seen in FIG. 5A. The needle 80 includes a sharpened distal tip 82 and a proximal grip 84 so that the needle can be advanced and retracted manually relative to the tubular body 62 of the catheter 60. The user can grab the grip 79 on the catheter 60 in one hand and the grip 84 at the proximal end of needle 80 in the other hand and simply advance and retract the needle by moving the grips together and apart.

At least one tension anchor 90 is reciprocatably disposed in each of the peripheral passages 70 so that the anchor may be retracted fully within the passage, as shown in FIG. 5A, or advanced distally beyond the ports 72, as shown in FIG. 4. The distal portions 92 of each of the tension anchors 90 will be adapted to form a three-dimensional geometry when advanced beyond the ports 72, again as shown in FIG. 4. In the illustrated embodiments, the three-dimensional geometry is a simple everting hook which bends back on the main shaft of the tension anchor by an angle of approximately 140-160°. A variety of other self-deploying and actively deployable anchors would also be suitable, such as malecot structures, levers, T-tags, balloons, barbs, screw-like structures and the like. The purpose of the three-dimensional structure is to have a narrow profile in which it can be advanced readily through the luminal walls (as described in more detail below) as well as an enlarged profile which is used to draw back on the posterior surface of the second luminal wall in order to apply tension by pulling proximally on the tension anchor.

In the exemplary embodiment, the tension anchor 90 is illustrated as a wire with a tissue penetrating distal tip. The distal portion 92 of the wire is pre-shaped to evert once tension anchor 90 is deployed beyond a preset distance from ports 72 of catheter 60. For example, the wires may be made of nitinol or other shape memory wire having the desired hook configuration preset so that in the wires are initially constrained in a straight configuration by peripheral passages 70 of catheter 60, as shown in FIG. 5A. After distally advancing the tension anchors 90 a predetermined distance, the wire will assume the hooked or everted configuration shown in FIG. 4. Said predetermined distance is approximately equal to the sum of the thickness of LW1, LW2 and the distance from peripheral passage port 72 and the anterior surface of LW1, the total typically being in the range of 4 mm to 10 mm. In this way, the region of the wire 90 which deflects remains within the passage 70 while the tip remains straight and advances distally through the tissue layers LW1 and LW2. Only after the deflection region passes through both tissue layers, will the tip of the wire be free from constraint so that it can evert into the hook. A particular advantage of the use of such shape memory materials is that they can be adapted to provide for a fixed level of tension when they are pulled back against the luminal walls being penetrated. When it is desired to remove the tension anchors, however, the tension above the fixate point can be applied so that the wires can be withdrawn after the desired treatment protocol has been completed.

Referring now to FIGS. 5A-5I, use of the catheter 60 for delivering the stent 86 across a first luminal wall LW1 of a first body lumen L1 and a second luminal wall LW2 of a second body lumen L2 will be described. The catheter 60 is introduced to the first body lumen L1, typically using an endoscope as described previously. The distal end 66 of the catheter 60 is aligned with and engaged against a target location 96 on an anterior surface of the wall LW1 of the first body lumen L1, as shown in FIG. 5A. Needle 80 can then be manually advanced by pushing grip 84 forwardly relative to the catheter body 62 while the user holds grip 79 on the catheter, resulting in the sharpened tip 82 penetrating both luminal walls LW1 and LW2 as shown in FIG. 5B. The tension anchors 90 will then be manually advanced by pushing on grips 98 at their proximal ends to advance them in their straightened configuration through the luminal walls LW1 and LW2, as shown in FIG. 5C. After being advanced through the walls by a predetermined distance sufficient to accommodate bending of the wire, the distal regions 92 are caused to assume a hooked or everted configuration, as shown in broken line in FIG. 5C.

After the tissue anchor wires 90 have entered into the second body lumen L2 and have been caused to assumed a hooked or everted configuration 92, as shown in FIG. 5C in broken line, the tension anchor wires 90 are drawn proximally to pull the hook structures 92 against the posterior wall LW2 and producing proximal tension which holds LW2 in close apposition to LW1 and to distal catheter end 66. The everted hooks may penetrate back through the posterior surface of the second luminal wall LW2 and to exit through the anterior surface of the first luminal wall LW1, as shown in FIG. 5D or they may remain inside LW2 and engage the surface or just under the surface of the posterior surface of LW2. While tension continues to be applied on the tissue anchor wires 90 to hold the posterior and anterior surfaces of the second luminal wall LW2 and the first luminal wall LW1 together, as shown in FIG. 5D, the tapered dilator tip 74 of the dilator assembly can be advanced over the needle 82 and through the tissue layers to dilate and enlarge the passage therethrough, as shown in FIG. 5E. The continuous tension applied by the tension anchor wires 92 will hold the luminal walls together, thus inhibiting fluid loss from either body lumen L1 or L2.

After the dilator tip 74 has been advanced through the luminal walls LW1 and LW2, the distal end 66 of the tubular body 62 of catheter 60 may be advanced through the tissue layers, as shown in FIG. 5F. The diameter of the distal end 66 will be made only slightly greater than that of the diameter of the proximal portion of the tapered distal tip 74, thus facilitating advancement of the catheter.

Once the distal end 66 of the tubular body 62 of catheter 60 is in place, as shown in FIG. 5F, the hollow shaft 76 of the dilator assembly can be advanced to release the stent 86 from constraint. As shown in FIG. 5G, a distal end of the stent 86 is first released and deployed radially outwardly. After the distal end has been deployed, as shown in FIG. 5G, the entire catheter assembly may be proximally retracted to engage the distal flange of stent 86 against the posterior surface of the second luminal wall LW2, as shown in FIG. 5H. As the stent will now be applying tension on the luminal walls, the tension anchors 92 may be withdrawn. After withdrawing the tension anchors, the distal end 66 of the tubular body 62 of the catheter 60 may be further retracted relative to the hollow shaft 76 to release the proximal portion of the stent, thus allowing the stent to fully deploy and capture the luminal walls LW1 and LW2, as shown in FIG. 5I. At that point, the entire catheter assembly may be withdrawn through the endoscope and the procedure is complete. The tension anchors may also be withdrawn following deployment of both distal and proximal portions of the stent.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for advancing a dilator distally through apposed luminal walls of adjacent first and second body lumens at a target site on an anterior surface of a first luminal wall, said method comprising:
deploying a tension anchor through the apposed luminal walls at a location laterally offset from the target site;
drawing the tension anchor proximally to hold a posterior surface of the first luminal wall against an anterior surface of a second luminal wall; and
advancing the dilator through the walls at the target site while continuing to hold the luminal wall surfaces together, wherein the dilator creates an enlarged passage.

2. A method as in claim 1, wherein the first body lumen is selected from the group consisting of the esophagus, the stomach, the duodenum, the small intestines, and the large intestines and the second body lumen is selected from the bile duct, the pancreatic duct, the gall bladder, cysts, pseudocysts, abscesses, the pancreas, the liver, the urinary bladder, duodenum, jejunum, and colon.

3. A method as in claim 1, wherein deploying comprises advancing a tether having a self-deploying anchor from the first body lumen, through the apposed luminal walls at the laterally offset location, and into the second body lumen, wherein the anchor self-deploys as tension is applied to the tether.

4. A method as in claim 3, wherein the tether having a self-deploying anchor comprises a T-bar anchor.

5. A method as in claim 4, wherein the T-bar anchor is initially disposed in a hollow needle, the needle is advanced from the first body lumen into the second body lumen, the T-bar anchor released from the needle to deploy in the second body lumen, and the needle withdrawn through the luminal walls.

6. A method as in claim 1, wherein deploying comprises positioning a distal end of a catheter through the first body lumen at the target site and advancing the tension anchor from the catheter through the apposed luminal walls.

7. A method as in claim 6, wherein a plurality of anchors are advanced from the catheter through the apposed luminal walls.

8. A method as in claim 6, wherein the anchor comprises a wire having a pre-shaped distal end, wherein the wire is advanced from an axial passage on the catheter so that the distal end assumes its shape upon entering the second body lumen and engages a posterior surface of the second luminal wall.

9. A method as in claim 6, wherein an access needle is advanced from a central passage of the catheter through the apposed luminal walls.

10. A method as in claim 9, wherein the access needle is advanced prior to advancing the tension anchor.

11. A method as in claim 9, wherein the access needle is advanced after advancing the tension anchor.

12. A method as in claim 9, wherein the dilator is advanced over the access needle to enlarge the passage through the apposed luminal walls.

13. A method as in claim 12, wherein the dilator has a cutting or electrosurgical tip.

14. A method as in claim 1, further comprising releasing a self-expanding stent within the enlarged passage.

15. A method as in claim 14, wherein the stent holds the first and second luminal walls together.

16. A method as in claim 14, wherein the stent is released from the dilator after the passage has been enlarged.

17. An apparatus for dilating a passage through apposed luminal walls, said apparatus comprising:
a catheter having a proximal end, a distal end, and a central passage therethrough;
a needle having a tissue penetrating distal tip, said needle being reciprocatably mounted in the central passage of the catheter so that the tissue penetrating distal tip can be advanced beyond said distal tip to penetrate the apposed luminal walls;
a dilator slidably mounted over the needle so that the dilator can be advanced to dilate the penetration formed by the needle through the apposed luminal walls; and
a tension anchor reciprocatably mounted on the catheter to penetrate a tissue location which is laterally offset from the dilator penetration location.

18. An apparatus as in claim 17, wherein the tension anchor is disposed in a peripheral lumen of the catheter.

19. An apparatus as in claim 18, comprising a plurality of tension anchors, wherein each tension anchor is disposed in a separate peripheral lumen of the catheter.

20. An apparatus as in claim 18, wherein the tension anchor comprises a wire having a pre-shaped distal end that is straightened when held in the peripheral lumen and which everts radially outwardly when it emerges from the peripheral lumen to hook into and engage the luminal walls.

21. An apparatus as in claim 17, wherein the dilator comprises a shaft having a tapered dilating tip at its distal end.

22. An apparatus as in claim 21, wherein the tapered dilating tip has a sharpened blade to cut tissue as it is advanced through the luminal walls.

23. An apparatus as in claim 21, further comprising a self-expanding stent carried on the dilator shaft proximal of the tapered distal tip, where said stent is constrained within a retractable tubular sheath.
